(19) United States
(12) Patent Application Publication
(10) Pub. No.: US 2002/0055696 A1
(43) Pub. Date: May 9, 2002

(54) Joint protective article

(75) Inventor: Bruno Borsoi, Victorio Veneto (IT)

Correspondence Address: GREENBLUM & BERNSTEIN, P.L.C., 1941 ROLAND CLARKE PLACE, RESTON, VA, 20191, US

(73) Assignee: SALOMON S.A, Metz-Tessy (FR)

(21) Appl. No.: 09/986,305

(22) Filed: Nov. 8, 2001

(30) Foreign Application Priority Data
Nov. 9, 2000 (FR) 0014668

(51) Int. Cl. A61F 5/00

(52) U.S. Cl. 602/32

(57) ABSTRACT

A protective article adapted to cover a joint by allowing the joint to bend. The protective article includes a frame that includes at least one bending zone having an abutment limiting the bending of the joint to a predetermined angle so as to avoid the hyper-flexion of the joint. The abutment is obtained by notches that are beveled so as to close for the predetermined angle. The protective article can especially be positioned on a boot in the area of the ankle.

### CROSS-REFERENCE TO RELATED APPLICATION

This application is based upon French Patent Application No. 00 14668, filed Nov. 9, 2000, the disclosure of which is hereby incorporated by reference thereto in its entirety, and the priority of which is hereby claimed under 35 U.S.C. §119.

### BACKGROUND OF THE INVENTION

1. Field of the Invention

The present invention relates to a protective article, adapted to cover a joint, and to protect the latter from excessive bending, with the least hindrance possible on the bending freedom of the joint over its usual bending range. In particular, the invention is adapted to equip a sports boot in order to protect the ankle joint, especially for sporting activities such as snowboarding, water skiing, Telemark skiing, in-line roller skating, and gliding on hand and guard rails.

2. Description of Background and Relevant Information

There are systems for flexible boots in the prior art for controlling the bending of the upper, and more specifically during snowboarding.

The invention described in the document EP 793 983 has a plate for controlling the bending of a snowboard boot, which is positioned on the top of the boot, between the instep and the base of the tibia. This plate, made of a rigid plastic material, has preferred bending folds and a specific geometry which provides it with a relative flexibility. However, it does not limit the hyper-flexions, i.e., when the angle between the tibia and the foot exceeds about 40° forwardly. This hyper-flexion, generating substantial stresses in the ankle, often cause fractures which often occur during jump landings. In fact, this plate stiffens the bending of the boot and distributes the tightening forces of the binding on the boot, without having a bending abutment.

The bending forces are also recovered by the rear spoiler of the binding, which is fixed without pivoting on the chassis, and which is connected to the control plate by a tightening strap.

There are also tongue reinforcements, for the liners, which are inserted in rigid boots, in particular for alpine skiing.

The invention described in the document EP 695 515 specifies a rigid tongue reinforcement that is movable on the liner to adjust its position, in relation to the rigid shell, to the user's foot. However, the bending is controlled by the rigid external shell and not by the reinforcement.

### SUMMARY OF THE INVENTION

An object of the present invention is to propose an article for protecting the joint, which has a bending abutment in order to prevent hyper-flexions of the ankle and to constitute a physiological abutment for the joint. In its abutting function, this bending abutment must be independent of any other additional element positioned around this joint.

Another object of the invention is to propose an embodiment in which the protective article is easily detachable, especially if it is associated with an envelope for the joint, such as a boot. Thus, the user can adapt his/her boot to the intended sporting activity. In addition, the protective article is advantageously compact so as to be housed in a garment pocket so that the user can carry it along during the sporting activity.

To achieve these objects, the protective article, which is adapted to cover a joint while allowing it to bend, includes a frame having at least one bending zone. This bending zone has an abutment that limits the bending of the joint for a predetermined angle so as to avoid the hyper-flexion of this joint.

The abutment made with notches, located in the bending zone, which are perpendicular or substantially perpendicular to the bending plane, and which are beveled such that the notches become closed for the predetermined bending angle.

In this position, the bending abutment is reinforced in that the frame is fixed on a flexible and substantially inextensible membrane that is located on the side of the abutment facing the joint.

The progressiveness of the bending is ensured by inserts made of compressible materials and positioned in the notches, in the area of the bending zone.

In a first embodiment, the protective article is detachably fixed on an envelope surrounding the joint to be protected. This first embodiment is defined in alternatives classified as families, namely, constructional alternatives and binding alternatives.

In a second embodiment, the protective article is non-detachably fixed on the envelope that surrounds the joint.

BRIEF DESCRIPTION OF DRAWINGS

The invention will be better understood, and other advantages thereof will become apparent from the description that follows, with reference to the annexed drawings, in which:

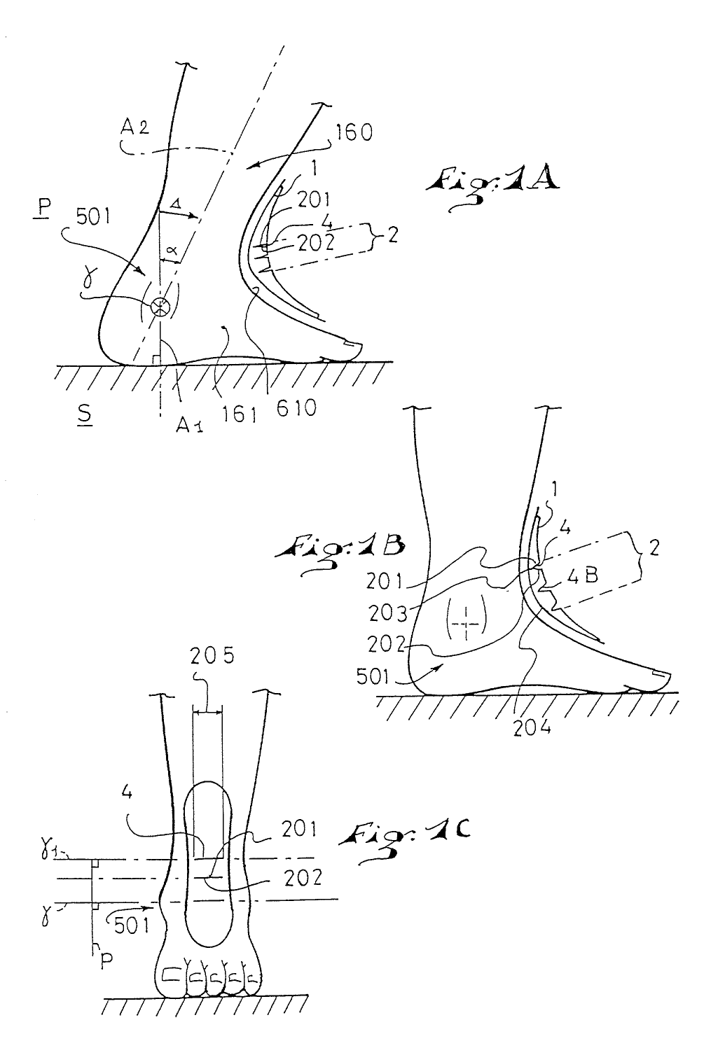

FIGS. 1A and 1B schematically show a side view of the ankle joint equipped with the protective article, the ankle being in the bent and unbent positions, respectively.

FIG. 1C schematically shows a front view of the ankle joint equipped with the protective article, the ankle being in the bent position.

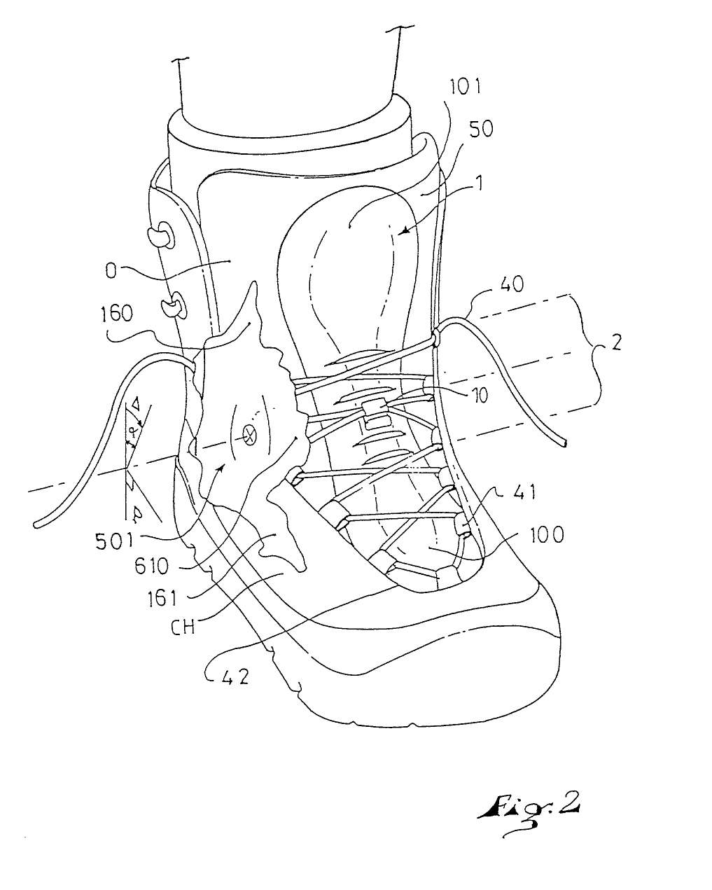

FIG. 2 shows a three-quarter front view of a boot equipped with the protective article according to the first embodiment. For convenience in understanding, the ankle area of the boot is shown broken-away.

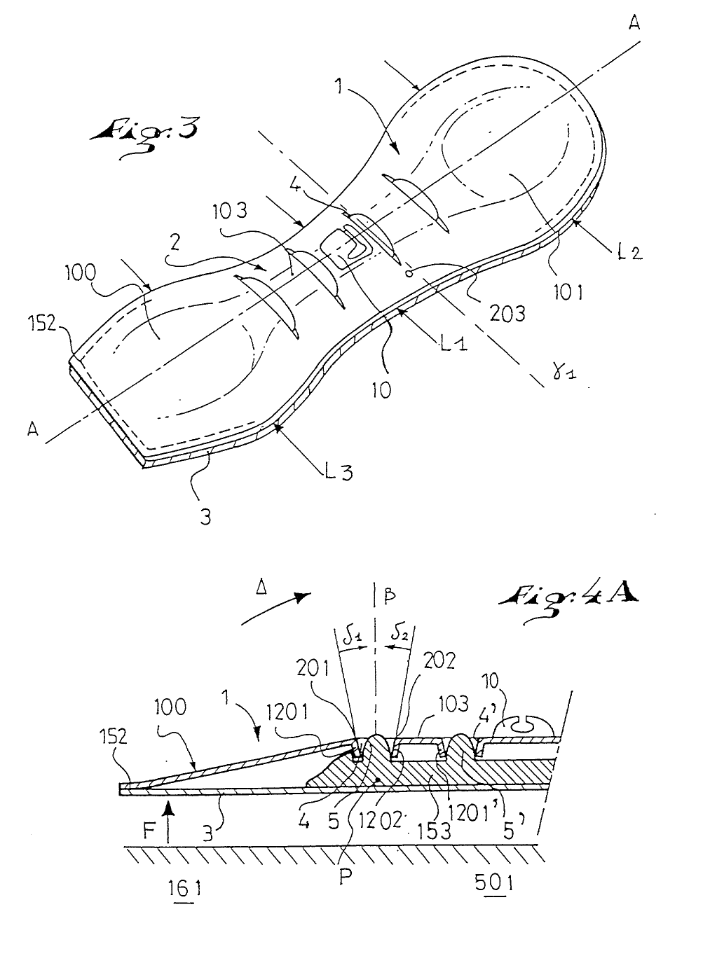

FIG. 3 shows a three-quarter top view of the protective article according to the first embodiment.

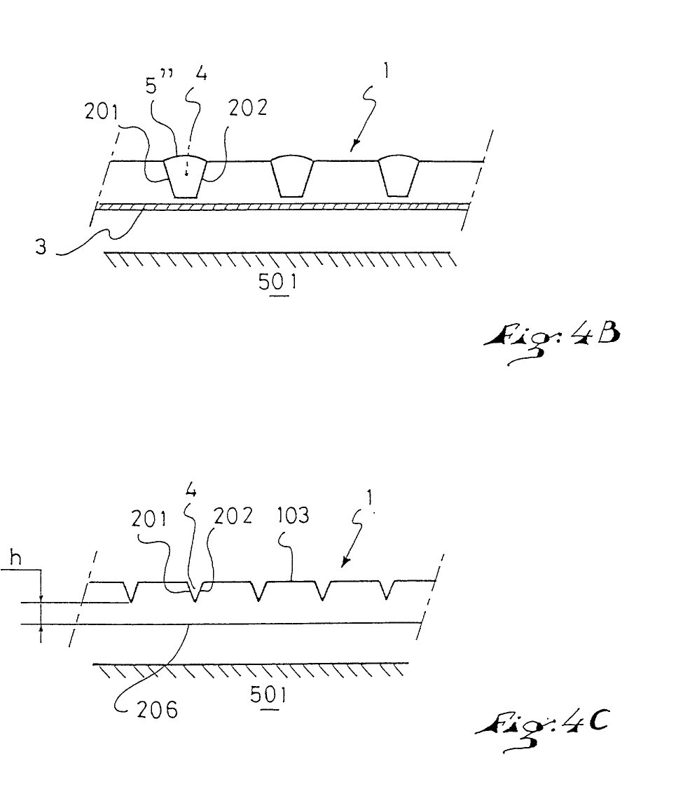

FIGS. 4A, 4B, and 4C schematically show cross-sections taken in the areas of arrows L1, L2, L3, respectively, of FIG. 3, for the protective article according to various constructional alternatives of the first embodiment.

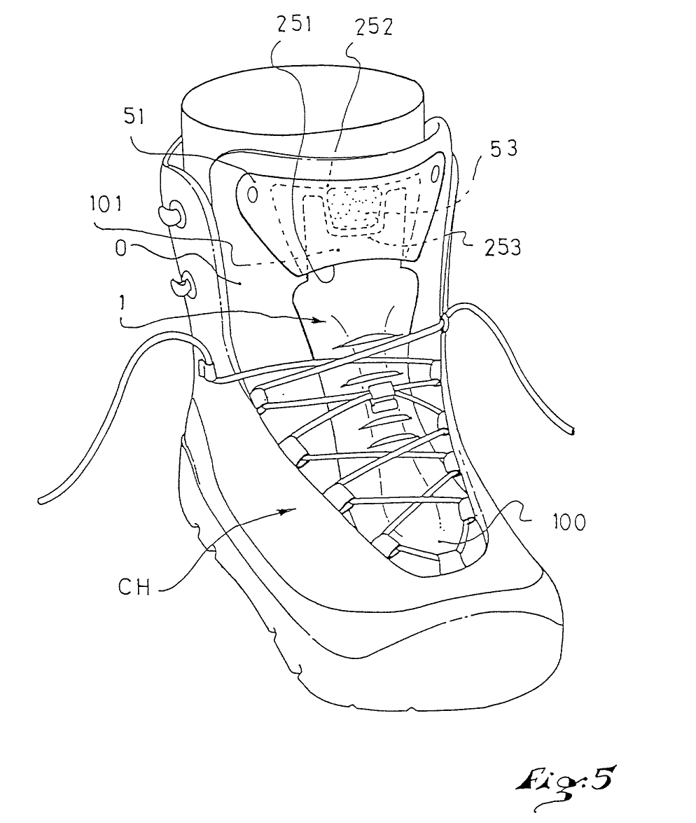

FIG. 5 schematically shows a three-quarter front view of a boot equipped with the protective article according to a first alternative assembly of the first embodiment.

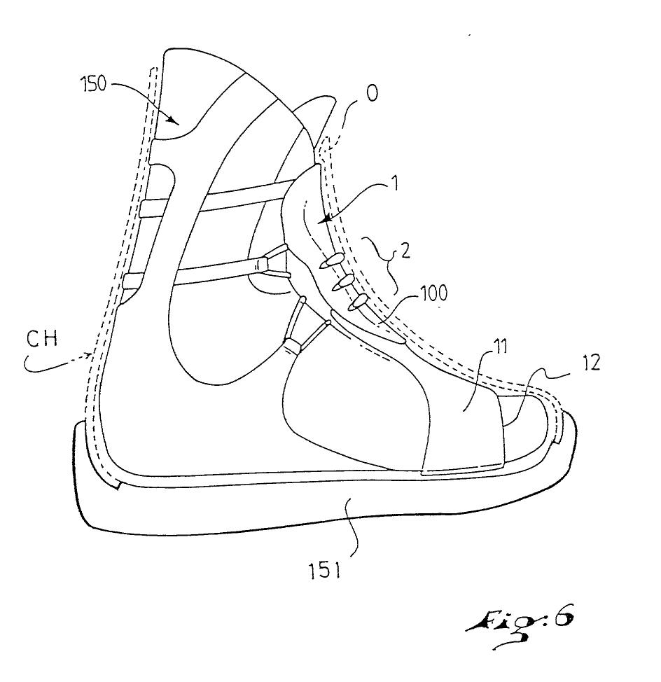

FIG. 6 schematically shows a side view of a boot, shown broken-away for convenience, showing an inner liner equipped with the protective article according to a second alternative assembly of the first embodiment.

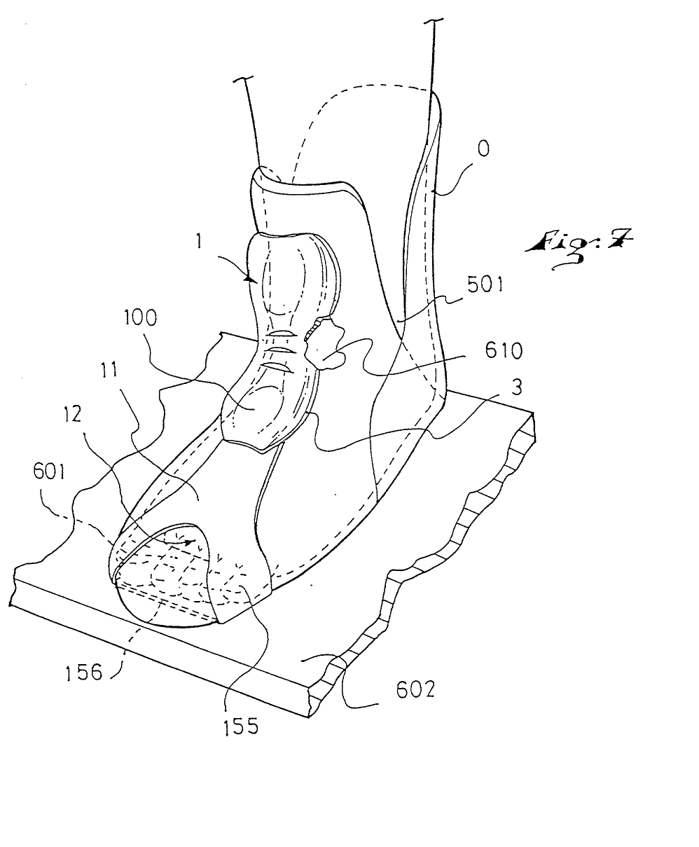

FIG. 7 schematically shows a three-quarter front view of a water ski binding equipped with the protective article according to the second embodiment.

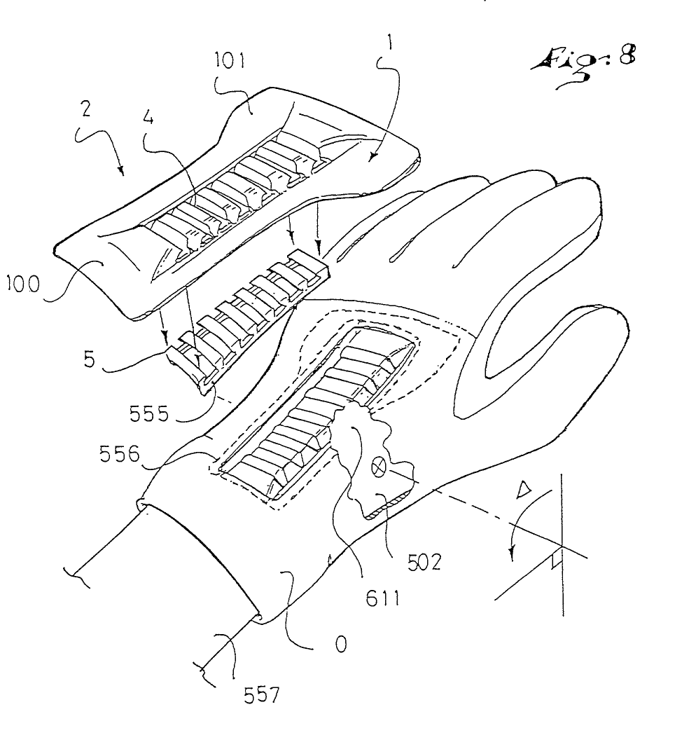

FIG. 8 schematically shows a three-quarter top view of a glove equipped with the protective article according to an alternative of the second embodiment.

### DETAILED DESCRIPTION OF THE INVENTION

FIG. 1A shows the ankle joint 501, located at the junction between the tibia 160 and the foot 161, which is shown in the bent position, in the bending plane P, along a direction Δ, oriented forwardly.

The ankle 501 is shown in maximum forward bending, before reaching the hyper-flexion of the joint 501. Such a hyper-flexion translates into abnormal forces on the bones of the foot 161 on the front surface 610, frequently causing fractures of the ankle joint 501.

The forward bending is referenced by an angle α, which quantifies the angle between the line A1 perpendicular to the foot support plane S and the axis A2 of the tibia 160, and by a bending axis γ that is substantially perpendicular to the plane P. Medical studies have shown that for an ankle 501, the bending angle α has a maximum value between 30° and 45°, depending on particular individuals.

The protective article covers the ankle joint 501 while allowing the latter to bend in the bending plane P which corresponds substantially to the physiological bending plane of the joint.

To avoid hyper-flexion, the protective article includes a rigid frame 1 having at least one bending zone 2. This bending zone 2 has an abutment that limits the bending of the joint 501 along the direction Δ for a predetermined angle α. Abutment is ensured during bending by at least one notch 4 positioned in the frame 1, in the bending zone. This notch 4 is beveled such that, for the predetermined angle α, the notch 4 becomes closed by bringing its edges 201, 202 in contact with one another. Once contact is made, it is the constituent material of the frame 1 itself that recovers the bending forces and, thus ensuring a firm abutment, stops the bending of the protective article and of the ankle 501.

In FIG. 1B, the ankle joint 501 is shown in the unbent position, the protective article allowing bending of the ankle 501 up to the abutting position. As shown previously, the bending freedom is also ensured by the bending zone 2 of the frame 1 in the bending range which is smaller than the predetermined angle. Indeed, the bending is concentrated more specifically in the area of the notches 4, 4B which, due to their beveled shape, have a relatively substantial range of movement for placing the edges 201, 202 in contact with one another. In fact, the overall bending is provided by the accumulation of localized bendings at each notch 4. Indeed, the closure of the notch 4 causes a localized bending in the area of a bending point 203 on the frame 1, which is located between the point of junction, of the edges 201 and 202 of the notch 4 and the inner surface 204 of the frame.

In FIG. 1C, the ankle joint 501 is in the bending position so as to place the edges 201 and 202 of the notch 4 in contact with one another, in the area of the bending zone 2. To constitute the most stable abutment possible, the geometry of the notch 4 is such that its edges 201 and 202 are in contact with one another along the entire width 205 of the notch 4. This optimum result is obtained for a notch 4 whose axis γ1 is substantially perpendicular to the bending plane P of the joint 501. This results in an axis γ1 substantially parallel to the bending axis γ of the joint 501, referenced in FIG. 1. Respecting this constructional arrangement also makes it possible to preserve a biomechanical compatibility between the frame 1 of the protective article and the joint 501, with respect to both the bending and the abutment. This compatibility is primordial since these two elements are going to cooperate with one another via an envelope, in particular of a boot or a glove.

The present description, with reference to FIGS. 1A, 1B, 1C, has been associated with the ankle 501 and with its journal axis γ; however, it is not exclusive to the ankle and remains applicable to any other joint of the body provided that the protective article is properly positioned on the side of the joint, where the bending occurs along the direction Δ. In the case of the ankle 501, the frame 1 of the protective article is located on the front surface 610 of the ankle 501, where the bending along the direction Δ brings the tibia 160 closer to the foot 161.

In FIG. 2, the frame I of the protective article is connected to the foot 161 and to the tibia 160 of the leg via a boot CH. As the bending abutment is ensured by the frame 1, it is necessary to properly make the connection between the frame 1 and the boot CH so that the bending abutment reverberates on the ankle joint 501.

Indeed, the foot 161, ankle 501, and tibia 160 are affixed to the boot CH by known tightening means 40, such as laces.

This type of physiological abutment, arranged on a boot, is particularly advantageous for sporting activities during which the athlete performs jumps, such as jumping off a springboard in snowboarding or in-line roller skating. Indeed, during jump landings, the athlete must control and adjust the necessary shock absorption obtained by the bending of the ankle and limit the amplitude of this same bending due to his/her muscles. Similarly, certain sports, such as Telemark skiing or ski jumping, cause the athlete to make genuflexions which combine the bending of the ankle and the bending of the knee and the control of this bending with the muscles.

In the preferred embodiment, shown in FIG. 2, the protective article including the frame 1 is detachable from the sports boot, enabling the user to adapt the boot to the sporting activity. Thus, the snowboarder can equip his/her boot with the device if it is desired to travel on any type of snow or on hilly ground, or the device can be removed if it is desired to travel along a track covered with packed snow. In this embodiment, the frame 1 is positioned outside the boot CH, in the area of the front portion of the envelope O which, in this case, is the upper O, and possibly the tongue 50 of the boot CH. As mentioned previously, it is imperative to firmly maintain the frame 1 on the boot CH. The frame includes supports 100, 101 on both sides of the bending zone 2. The front support 100 is positioned so as to be inserted, at the base of the tongue 50, between the tongue 50 and the lace keepers 41 as well as the lace 40.

To block the base of the frame 1 laterally, the geometry and especially the width of the front support 100 are preferably adapted to the geometry of the front portion 42 of the upper O in the area of the tongue 50. The forward blocking of the frame 1 in relation to the boot CH is obtained by fixing the tongue 50 on the front portion 42 of the upper I which can be obtained in particular by seams. Moreover, the frame 1 is maintained at the front by the action of the tibia 160 on the rear support 101 of the frame 1. As the frame 1 is more rigid than the upper O, the bending zone 2 must be blocked against the upper O in the area of the front surface 610 of the foot. This blocking function is achieved by at least one cooperation mechanism 10 located on the frame 1, complementary of the tightening means 40, such as laces of the boot. This cooperation mechanism 10, located in the area of the bending zone 2, also makes it possible to stabilize the frame 1 laterally in relation to the boot CH, while benefiting from the normal pressure exerted by the lace 40 which maintains the frame 1 flat against the tongue 50.

FIG. 3 shows the protective article in detail, once it has been removed from its support such as the boot. The frame 1 has a geometry that is extended on both sides of the bending zone 2 by supports 100, 101, along a given direction AA substantially perpendicular to the axis γ1 of the notches 4. These supports 100, 101 enable the frame 1 to take support on the parts of the body that extend on both sides of the joint to be protected, namely, the tibia and the instep for the ankle joint. To guarantee maximum comfort for the user, the supports 100, 101 are as wide as possible, in order to better distribute the pressures on these body parts. This arrangement translates into widths L2 and L3, along an axis Δ of the supports 100, 101, respectively, greater than the width L1 of the frame 1 in the area of the bending zone 2. Conversely, the bending zone 2, located between the supports 100, 101, makes it possible to control the bending. Since this bending is essentially concentrated in the area of the notches 4, the geometry of the frame 1 must limit the residual bendings of the frame 1 in the zones 103 between notches. A solution for achieving this objective is to retain a frame 1 that is thicker, in the zones 103 between notches, than the supports 100,101, and therefore has greater bending inertia.

To guarantee stability of the bending abutment, with respect to the bending load, the frame 1 is fixed on a membrane 3 that is flexible and substantially inextensible. The fixing is obtained by appropriate means such as seams 152 that extend at least along the ends of the supports 100, 101. The seams 152 can extend along the entire periphery of the frame 1.

FIGS. 4A, 4B, and 4C schematically show the protective article along the longitudinal cross-section AA positioned, in FIG. 3, in various alternatives of the first embodiment.

FIG. 4A shows, in a cross section taken along axis AA, the first embodiment already illustrated in the previous figure. The frame 1 is constituted by a shaped piece that is assembled by seams 152 on a membrane 3, which is itself opposite a part of the body 161. The frame 1 includes notches 4 that can close under a bending force along a direction Δ. This bending is generated by the force F exerted by a part of the body 161, located in the vicinity of the joint 501. Under the effect of the bending, the edges 201 and 202 of the notch 4 tend to come closer together by pivoting symmetrically along angles δ1 and δ2, until they are in contact with one another in a plane β substantially positioned along the bisectrix of the notch 4. In the preferred embodiment, presently shown, the frame 1 is made of a thermo-injected plastic material, such as polyamide, polyurethane, in particular, which combines a certain flexibility of the material with shapes which make it possible to obtain geometries having a good rigidity.

An insert 5, made of a compressible material, is positioned in the notch 4. This insert 5 is compressed by the closure of the notch 4. However, since the forces transmitted to the insert are substantial, the compression of the insert must be optimized so as to avoid premature aging of the material, especially by repeated punching cycles. Thus, the frame 1 is preferably extended in the notch 4 by walls 1201, 1202, which extend from the edges 201 and 202 in the notch 4, toward the virtual bending point P. This virtual bending point P corresponds to the projection, in the cross-sectional plane AA, of the localized bending zone 203 referenced in FIG. 3. Respecting this constructional arrangement makes it possible to obtain a shell structure for the zone 103 between notches. Indeed, the return walls 1202 and 1201 constitute stiffeners for the frame 1 in the area of the zone 103 between notches and guarantee a very substantial rigidity by practically eliminating bending in this zone.

To prevent the inserts 5, 5′, located in the notches 4, 4′, respectively, from escaping from the notch under compression, the inserts 5, 5′, are connected to one another by a main body 153. The main body 153 has an adequate thickness to substantially fill the inner empty space of the frame 1 and to position itself practically in contact with the membrane 3. The main body 153 is positioned between the frame 1 and the membrane 3.

During bending along the direction Δ, the membrane 3 is biased in traction, contrary to the inserts 5, 5′ which are biased in compression. However, the inextensible nature of the membrane 3 guarantees a firm bending abutment. Indeed, if the membrane 3 could deform, bending in the area of the abutment would then include a supplemental and unwanted component, caused by the elongation of the membrane 3 and of the portion of the frame 1 that is located in the vicinity of the membrane 3. Respecting this constructional arrangement therefore guarantees a controlled abutment of the bending by biasing the frame 1 in traction only.

For certain sporting activities, it can be advantageous to modify the hardness of the bending control provided by the inserts 5, 5′. One way to make the inserts 5, 5′ interchangeable, while preserving a definitive assembly of the frame 1 on the membrane 3, consists of positioning the assembly seams 152, arranged transversely, only at the ends of the supports 100. Thus, to release the inserts 5, 5′ and the main body 153, a counter-bending is applied on the frame 1 which frees a space between the frame 1 and the membrane 3 due to the absence of longitudinal seams.

FIG. 4B shows an alternative embodiment that keeps the substantially inextensible membrane 3 located on the side of the joint 501. The frame 1 still has a notch 4 that functions as previously described by bringing the edges 201 and 202 closer together. The notch 4 is also equipped with an insert 5″ made of a compressible material. However, the insert 5″ is affixed to the frame 1 by a chemical assembly means. The insert 5″, constituted of a different material than that of the frame 1, can simply be glued or bi-injection or sur-injection molded on the frame 1. With the latter technique, one must assume that the two different materials are plastics that are compatible with one another.

FIG. 4C shows a second alternative embodiment which does not have a membrane. It keeps the notch 4 which functions, for the abutment, by bringing the two edges 201 and 202 closer together. However, the notch 4 does not extend entirely through the frame 1 and allows a height h between the base of the notch 4 and the lower portion 206 of the frame 1. Since the frame 1 is preferably integral, the material of the portion located on the height h, which is opposite the joint 501, constitutes a zone that resists bending. It is this continuity of material, in the area of the lower surface 206, which provides progressiveness in the bending. Moreover, the material used must resist traction so as to ensure a firm bending abutment consistent with the functioning of the inextensible membrane previously described.

The frame 1 can be constituted of a continuous lower layer on which are assembled elements having at least one zone 103 between notches. The thickness of these elements can advantageously be equal to the height of the notches 4. Furthermore, this embodiment is very economical and possibly allows obtaining dual material frames.

FIG. 5 shows an improvement of the main embodiment previously described in FIG. 2. This improvement makes it possible to obtain a connection between the top of the frame 1 and the envelope O, which is also the upper O of the boot CH. However, the frame 1 remains detachable with respect to the upper O. The object of this improvement is to use the differential vertical sliding that occurs during the forward bending of the upper O, between the front of the upper O and the rear support 101. Indeed, during bending, the top of the frame 1 tends to rise slightly along the front of the upper O, despite a good positioning of the frame 1 on the upper O. The idea is to store the energy produced by this displacement and to return it by restarting the maneuver when bending stops. To achieve this object, the front surface of the upper includes, in its upper portion, a pocket 51 that is affixed to the upper O and which has an opening 251 located toward the bottom. In this pocket 51 is inserted, through the opening 251, the rear support 101 that cooperates with a shock absorbing element 53. This shock absorbing element 53, positioned in the pocket 51, is compressed against the upper portion 252 of the pocket 51 by the rear support 101.

The energizing function obtained by compression of the element 53 also makes it possible to improve the bending control, in particular when the bending gets close to the predetermined maximum angle. Indeed, the differential sliding between the upper O and the frame 1 occurs for a quite pronounced bending.

This improvement can be applied generally to any envelope that surrounds the joint. In addition, the shock absorbing element can be compressed by the front support 100 of the frame 1. This element is then positioned at the front of the boot, at the front of the frame 1. Both arrangements of the energizing functions can be combined.

In FIG. 6, the frame 1 is positioned within the envelope O, which is also the upper O of the boot CH, between the upper O and the inner liner 150. However, since the inner liner 150 is very flexible, it is not capable of distributing the pressure exerted by the front support 100 on the instep. This support would painfully crush the foot during bending. This is why it is preferable to fix the front support 100, which is positioned beneath the bending zone 2, on a shell 11. This rigid shell 11 takes supports on the sole 151 of the boot CH and thus transmits the supports of the frame 1 directly on the sole 151 by creating a vault effect above the toes.

The shell 11, which combines rigidity and thinness in order to reduce the space requirement, can advantageously be made of a thermoformed material of the plastic type, in particular polyamide, polyethylene, polyurethanne, according to a shell having a thickness within, or substantially within, a range of 0.5-3.0 millimeters (mm).

In addition, the shell 11 can advantageously include a recess 12 in the area of the toes to guarantee a better comfort for the forefoot portion.

In this embodiment, the protective article remains detachable. The user can remove from or replace in his boot CH the protective article which is affixed on the shell 11.

FIG. 7 shows the protective article associated with a binding assembly adapted to fix the foot 601 on a gliding apparatus 602. The device shown is a binding constituted of an envelope O which surrounds the foot 601 and the ankle 501, and which is fixed on an apparatus 602 adapted for boat-drawn waterskiing.

Since the object is still to obtain a forward bending abutment, the protective article is arranged on the front surface 610 of the foot, in the area of the ankle 501. Since the envelope O is very flexible and generally made of neoprene, it is advantageous to use a shell 11 that allows a recess 12 in the area of the toes, thus creating lateral bands 155 arranged substantially symmetrically with respect to the foot.

In this second embodiment, the protective article is fixed permanently on the envelope O of the binding. The frame 1 is fixed on the neoprene by appropriate means such as seams. However, since the neoprene is extensible, it is necessary to keep the membrane 3 inextensible. This membrane 3 is maintained inserted between the frame 1 and the envelope O. Similarly, the shell 11, connected to the support 100, is fixed on the gliding apparatus 602 via at least one lug 156 which can be beneath the foot or outside the binding.

The invention is applicable to detachable protective articles. In such a case, the previously disclosed embodiments shown on a boot are applicable to the present binding.

FIG. 8 shows the protective article non-detachably associated with a glove. During the sporting activity, the wrist 502 is often biased by bendings along the rearward direction Δ. These bendings for opening the wrist 502 outwardly, which can be extreme and very intense, in particular during falls, frequently cause sprains of the wrist. This calls for the necessity to position the frame 1 of the protective article in the area of the wrist 502, on the top of the hand 611. If the envelope O, which constitutes a portion of the glove, located on the top of the hand 611, is flexible and substantially inextensible, the envelope O can advantageously be used as a membrane in the protective article. This means that the frame 1, which includes notches 4 and incorporates inserts 5 connected by a chassis 555, is fixed directly on the envelope O of the glove by appropriate means such as seams 556.

In addition, the frame 1 can advantageously have a rear support 101 that is wider than the bending zone 2 in order to distribute the supports toward the fingers. Similarly, the front support 100 of the frame I may not be important and can have a width substantially identical to that of the bending zone 2. Indeed, the wrist 502 and the forearm 557, on which the support 100 is supported, have widths that are substantially equal.

Other applications of the present invention, not shown, can be envisioned. In a non-exhaustive manner, the protective article can possibly be positioned in the area of the knee, the frame being arranged on the front surface of the leg, or at the rear of the knee. Using this area makes it possible to prevent sprains and ruptures of knee ligaments caused by rear hyper-flexions, in particular during alpine skiing. Similarly, one can envision using the protective article on the spine, the frame being arranged in the back, in the area of the neck. This arrangement protects the nape of the neck from the “whiplash” caused by a rear impact which causes a rearward hyper-flexion of the neck.

The present invention is not limited to the embodiments described hereinabove, which are provided for guidance only, but encompasses all similar or equivalent embodiments. Thus, in each case, the protective frame can be either fixed or detachable.

What is claimed is:

1. A protective article for a joint of a person's body, said article comprising:
a rigid frame adapted to cover the joint, said rigid frame having a flexibility to allow bending of the protective article along at least one bending direction, said rigid frame comprising at least one bending zone having an abutment limiting bending of the joint along said bending direction at a predetermined angle so as to avoid hyper-flexion of the joint.

2. A protective article according to claim 1, wherein the bending zone includes at least one notch substantially perpendicular to a bending plane, and wherein the notch is beveled to become closed for said predetermined bending angle so as to constitute said abutment.

3. A protective article according to claim 1, wherein the frame is fixed on a flexible and substantially inextensible membrane located on a side of the joint.

4. A protective article according to claim 2, wherein said bending zone includes an insert constituted of a compressible material positioned in said notch.

5. A protective article according to claim 2, wherein said rigid frame is extended on opposiete sides of the bending zone by supports, and wherein said bending zone is thicker and thinner than said supports.

6. A protective article according to claim 5, further comprising an envelope, and wherein at least one support cooperates with a shock-absorbing element affixed to said envelope for improving control of the bending by compression.

7. A protective article according to claim 6, wherein said shock-absorbing element is positioned in a pocket affixed to said envelope.

8. A boot incorporating a protective article according to claim 1, wherein said rigid frame is positioned, in an area of an ankle of a foot, on a front surface of the foot.

9. An assembly for binding a foot to a sports apparatus incorporating the protective article according to claim 1, wherein said rigid frame is positioned, in an area of an ankle of a foot, on a front surface of the foot.

10. A glove incorporating the protective article according to claim 1, wherein said rigid frame is positioned, at an area of a wrist of a hand, on a top of the hand.

11. A protective article according to claim 1, wherein said rigid frame is positioned, in an area of a knee, on a front surface of the leg.

12. A protective article according to claim 1, wherein said rigid frame is positioned on a spine and especially in an area of the neck, on the back.

13. A boot according to claim 8, wherein the boot includes a boot-tightening means, and wherein said rigid frame includes at least one cooperating mechanism complementary of said boot-tightening means.

14. A boot according to claim 8, wherein the boot includes a shell supported on a sole, and wherein said front portion is fixed on said shell.

15. A boot according to claim 14, wherein said shell includes a recess in an area for accommodating toes of a wearer.

16. A protective article according to claim 1, wherein said predetermined angle, for an ankle, has a value comprised between +30° and +45°.
